(19) United States
(12) Patent Application Publication
(10) Pub. No.: US 2006/0252820 A1
(43) Pub. Date: Nov. 9, 2006

(54) Composition for oral administration containing alkylene dioxybenzene derivative

(76) Inventor: Tetsuya Suzuki, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/554,205

(22) PCT Filed: Apr. 23, 2004
(86) PCT No.: PCT/JP04/05864

§ 371 Date: Dec. 12, 2005

(30) Foreign Application Priority Data
Apr. 25, 2003 (JP) 2003121339

(51) Int. Cl. A61K 31/355; A61K 9/24

(52) U.S. Cl. 514/452; 424/472

(57) ABSTRACT

The present invention relates to a composition for oral administration containing an alkylenedioxybenzene derivative represented by the general formula (I): (wherein m represents an integer of 2 to 5, and n represents an integer of 1 to 3) or an acid addition salt thereof, which allows for duration of the drug concentration in plasma. By dispersing a compound represented by the general formula (I) in a matrix containing a synthetic polymer and/or waxes, a safe and useful pharmaceutical preparation for oral administration having the stable drug releasing property in a living body was obtained.

### TECHNICAL FIELD

The present invention relates to a composition for oral administration which releases a drug at a controlled releasing rate when orally administered.

### BACKGROUND ART

Generally, the drug concentration in blood is used as one index of drug efficacy, undesirable reaction (side effect, allergic reaction, excessive reaction), and many examples are reported in which occurrence of an undesirable reaction depends on the drug concentration in blood. In order to avoid these undesirable reactions and improve QOL of a patient, it is essential to control the drug concentration in blood.

When dose dependency is observed between the drug concentration in blood and a undesirable reaction, prolongation/regulation of release of a drug from a drug preparation is frequently performed in order to alleviate an undesirable reaction. Design of these regulated/prolonged release compositions and techniques of controlling a releasing rate have been developed by those skilled in the art for a few decades, and are well-known in the art (L. Krowczynski, Extended Release Dosage Forms, CRC-Press Inc., USA, 1987).

An alkylenedioxybenzene derivative represented by the general formula (I): 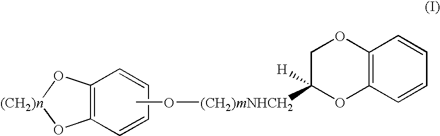
(wherein m represents an integer of 2 to 5, and n represents an integer of 1 to 3)
or an acid addition salt is a compound which has the strong affinity for a 5-HT_{1A} receptor, and is expected as a drug for treating anxiety, for manic-depressive psychosis (U.S. Pat. No. 5,168,099).

When a conventional preparation (a tablet using D-mannitol as an excipient, sodium carboxymethylstarch as a disintegrating agent, and hydroxypropylcellulose as a binder) of 5-[3-[[(2S)-1,4-benzodioxan-2-ylmethyl]amino]propoxy]-1,3-benzodioxole hydrochloride (hereinafter, referred to as MKC-242 in some cases in the present specification) which is one of an alkylenedioxybenzene derivative represented by the general formula (I) or an acid addition salts is administered to a human, symptoms such as nausea, dizziness, orthostatic syncope and the like are recognized although they are all slight, and it has been found that these undesirable symptoms occur depending on the drug concentration in plasma. In addition, a half-life T_{1/2} is as short as 2 to 5 hours, and the drug concentration in plasma does not last.

On the other hand, the solubility of a drug is pH-dependent and, in view of the physical and physiological environment in a digestive tract of a human and a digestive tract moving time of a preparation, it is considered that an alkylenedioxybenzene derivative is not suitable for an oral regulated/prolonged release dosage form.

An object of the present invention is to overcome these problems of kinetics in a living body and pharmacological activity or physicochemical drawbacks possessed by an alkylenedioxybenzene derivative or an acid addition salt thereof, and provide a composition for oral administration in which the drug concentration is not rapidly increased in plasma, and the drug concentration in plasma can be maintained over a long period of time.

### DISCLOSURE OF INVENTION

The present inventors found that the problems can be solved by dispersing a compound of the general formula (I) in a matrix material, or coating a composition containing the compound of the general formula (I) with an enteric film, or combining both of them.

The present invention relates to a composition for oral administration which releases a drug at a controlled releasing rate when orally administered. That is, the present invention provides a composition for oral administration which suppresses rapid increase of the drug concentration in plasma of an alkylenedioxybenzene derivative represented by the general formula (I):

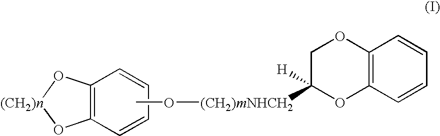

(wherein m represents an integer of 2 to 5, and n represents an integer 1 to 3)

or an acid addition salt thereof, and allows for duration of the drug concentration in plasma.

### BRIEF DESCRIPTION OF DRAWINGS

FIG. 1 shows the results of a dissolution test of the composition obtained in Example 1.

FIG. 2 shows the results of a dissolution test of a conventional preparation obtained in Comparative example 1.

### BEST MODE FOR CARRYING OUT THE INVENTION

The present invention is as follows:
(1) A composition for oral administration containing an alkylenedioxybenzene derivative represented by the general formula (I) or an acid addition salt thereof and a matrix material and/or a coating material.
(2) The composition described in the above (1), wherein each of the material and the coating material is at least one kind selected from a synthetic polymer and waxes.
(3) A composition for oral administration, wherein an alkylenedioxybenzene represented by the general formula (I) or an addition salt thereof is dispersed in a matrix containing a synthetic polymer and/or waxes.
(4) A composition for oral administration, characterized in that a composition containing an alkylenedioxybenzene derivative represented by the general formula (I) or an acid addition salt thereof and a synthetic polymer and/or waxes is coated with a coating agent containing a synthetic polymer.
(5) A composition for oral administration containing an alkylenedioxybenzene derivative represented by the general formula (I) or an acid addition salt thereof in a matrix containing waxes and an excipient.
(6) A composition for oral administration, wherein a base granule containing an alkylenedioxybenzene derivative represented by the general formula (I) or an acid addition salt thereof dispersed in a matrix containing waxes and an excipient is coated with an enteric film.
(7) The composition for oral administration described in the above (6), wherein an amount of waxes is 5 to 70% by weight relative to the base granule.
(8) The composition for oral administration described in any one of the above (1) to (6), wherein the synthetic polymer is at least one kind selected from polyvinyl type, acrylic acid or acrylic acid ester type and cellulose type, and the waxes are at least one kind selected from shellac, gelatin, hydrogenated oil, higher fatty acid and esters thereof, higher aliphatic alcohol, and natural and synthetic waxes.
(9) A capsule containing an alkylenedioxybenzene derivative or an acid addition salt thereof, comprising the composition for oral administration described in the above (1) filled therein.
(10) A process for preparing a composition for oral administration, comprising kneading an alkylenedioxybenzene derivative represented by the general formula (I) or an acid addition salt thereof, waxes and an excipient to obtain a granule, and coating the granule with an enteric film.
(11) A composition for oral administration, wherein a pharmaceutically active substance is an aklylenedioxybenzene derivative represented by the general formula (I) or an acid addition salt thereof, and a time for releasing at least 80% of a content of the pharmaceutically active substance is 2 to 24 hours when tested at 100 rotations per minute using 900 ml of a hydrochloric acid/trisodium phosphate buffer (pH 6.8) according to a basket method (USP dissolution test method first method).
(12) The composition for oral administration according to any one of the above (1) to (11), wherein the alkylenedioxybenzene derivative or the acid addition salt thereof is 5-[3-[[(2S)-1,4-benzodioxan-2-ylmethyl]amino]propoxy]-1,3-benzodioxole hydrochloride.

Examples of the alkylenedioxybenzene derivative represented by the general formula (I) include compounds represented by Table 1 and Table 2.

TABLE 1

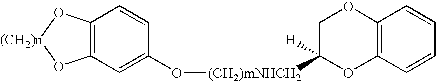

| Compound No. | M | n |
|---|---|---|
| 1 | 3 | 1 |
| 2 | 3 | 2 |
| 3 | 3 | 3 |
| 4 | 4 | 1 |
| 5 | 4 | 2 |
| 6 | 4 | 3 |
| 7 | 5 | 1 |
| 8 | 5 | 2 |
| 9 | 5 | 3 |
| 10 | 2 | 1 |
| 11 | 2 | 2 |
| 12 | 2 | 3 |

TABLE 2

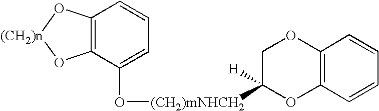

| Compound No. | M | n |
|---|---|---|
| 13 | 3 | 1 |
| 14 | 3 | 2 |
| 15 | 3 | 3 |
| 16 | 4 | 1 |
| 17 | 4 | 2 |
| 18 | 4 | 3 |
| 19 | 5 | 1 |
| 20 | 5 | 2 |
| 21 | 5 | 3 |
| 22 | 2 | 1 |
| 23 | 2 | 2 |
| 24 | 2 | 3 |

Of the compounds represented by the general formula (I), a compound of Compound No. 1 is preferable. Examples of an acid in the acid addition salt of the alkylenedioxybenzene derivative include inorganic acids such as hydrochloric acid, hydrobromic acid, sulfuric acid, phosphoric acid and nitric acid, and organic acids such as acetic acid, succinic acid, adipic acid, propionic acid, tartaric acid, fumaric acid, maleic acid, oxalic acid, citric acid, benzoic acid, toluenesulfonic acid and methanesulfonic acid. The alkylenedioxybenzene derivative of the general formula (I) or an acid addition salt thereof can be synthesized by the method described in U.S. Pat. No. 5,168,099.

Since the present invention is a dosage form that at least 80% of a content of a drug is released in 2 to 24 hours in a USP dissolution test method first method, it can be applied to all kinds of regulated-release compositions which are known in the art.

Further, the present invention provides a composition for oral administration comprising the following (a) and (b).
(a) An alkylenedioxybenzene derivative of the general formula (I) or an acid addition salt thereof.
(b) At least one release rate-controlling substances having the effects showing the following (1) and/or (2) by administering the composition to a patient:
   (1) a time to a maximum plasma concentration (Tₘₐₓ) of the alkylenedioxybenzene derivative of the general formula (I) or the acid addition salt thereof ranging from 1.5 to 4.5 hours following administration; and
   (2) Cₘₐₓ in plasma, of the alkylenedioxybenzene derivative of the general formula (I) or the acid addition salt thereof is 100 to 300 ng/ml.

A matrix material and a coating material used may be any materials as far as they prevent permeation of water into a composition, and can control a desired releasing rate of a drug, and examples thereof include a synthetic polymer and waxes. It is preferable that amounts of a synthetic polymer and waxes to be used are 5 to 70% by weight, preferably 20 to 50% by weight relative to a base granule or a base tablet.

Preferable examples of the synthetic polymer include polyvinyl type (polyvinylalcohol, polyvinylpyrrolidone etc.), acrylic acid or acrylic acid ester type (polymer of methyl methacrylate or copolymer of acryl monomer, e.g. methacrylic acid copolymer LD, methacrylic acid copolymer L, methacrylic acid copolymer S, aminoalkyl methacrylate copolymer RS etc.), and cellulose type (biopolymer or degenerated biopolymer of cellulose, e.g. ethylcellulose, cellulose acetate phthalate, hydroxypropylcellulose, hydroxypropylmethylcellulose, methylcellulose, sodium carboxymethylcellulose etc.).

Examples of the waxes include shellac, gelatin, hydrogenated oil (fat obtained by adding hydrogen to vegetable or animal fatty oil, such as hydrogenated beef tallow, hydrogenated castor oil, hydrogenated cottonseed oil, hydrogenated soybean oil etc.), higher fatty acid and esters thereof (stearic acid, palmitic acid, alminium monostearate, glyceryl mono- or dipalmitate, glyceryl mono-, di- or tristearate etc.), higher aliphatic alcohol (cetyl alcohol, stearyl alcohol, myristyl alcohol, 12-hydroxystearyl alcohol etc.), and natural and synthetic waxes (beewax, Japan wax, carnauba wax, paraffin wax, whale wax, synthetic wax etc.). Among them, hydrogenated oil is preferable.

These synthetic polymers and waxes may be used alone or in combination.

In the composition of the invention, various additives which are normally used in the art such as excipient, binder, lubricant and aggregation preventing agent can be used. Examples of the excipient include sugars (white sugar, lactose, glucose, maltose etc.), sugar alcohol (mannitol, sorbitol, xylitol etc.), starch, crystalline cellulose, calcium phosphate and calcium sulfate. Examples of the binder include polyvinyl alcohol, polyacrylic acid, polymethacrylic acid, polyvinylpyrrolidone, dextrin, hydroxyethylcellulose, hydroxypropylmethylcellulose, hydroxypropylcellulose, macrogols, gum arabic, gelatin, agar and starch. In addition, examples of the lubricant and aggregation preventing agent include talc, magnesium stearate, calcium stearate, and polyethylene glycols. These can be used by appropriately combining them.

A granule can be prepared by adding a synthetic polymer or waxes to a drug and, if necessary, adding an excipient such as lactose and mannitol, adding a solvent, and kneading them to be granulated and dried (Process 1). Since, in the thus obtained granule, the drug is dispersed in a matrix containing the synthetic polymer or waxes, the drug is gradually released in water or in a gastrointestinal tract.

The granule in the present invention can be also obtained by adding an excipient such as lactose and mannitol to a drug, adding small amounts of a binder and a solvent, and granulating to prepare a normal granule which is not dispersed into a matrix, and coating this granule with an enteric firm. Also in this case, the drug is gradually released in an intestinal tract. These granulation and coating can be performed by apparatuses which are conventionally used.

pH in stomach is usually 1.8 to 4.5 in a healthy subject, and in view of dependency of the solubility of the alkylenedioxybenzene derivative on a pH, stable drug release in stomach can not be desired. In view of that a pH variation in intestine is 6.5 to 7.5, after passed through stomach having a great pH variation due to enteric firm coating, stable release becomes possible in an intestinal tract. Therefore, it is also possible to coat the composition obtained in the aforementioned process 1 with an enteric film.

A preferable aspect of the present invention is to mix an alkylenedioxybenzene derivative and a synthetic polymer and/or waxes, and an additive, extrude and granulate the mixture, and coat the resulting granule with an enteric film. Further, by filling the thus obtained regulated/prolonged release composition into a capsule, a capsule agent can be provided.

As the enteric film, the aforementioned synthetic polymer and waxes can be used. A method of coating with an enteric film in not particularly limited. Any of aqueous and non-aqueous systems which are conventionally used in this field can be applied, and application can be performed by methods which are conventionally used in the pharmacy techniques such as spray coating in a fluidized bed coating method, a rolling flowing type coating method, and the like.

A coating rate of the enteric film is different depending on a kind of a drug or film and, in each case, the rate may be appropriately regulated. It is desirable to use a film such that a coating amount becomes 5 to 50 W/W % relative to a drug. Preferably, a film is used at an amount of 20 to 40% by weight relative to a granule, at an amount of 5 to 30% by weight relative to a tablet.

### EXAMPLES

The present invention will be explained in more detail below by way of Examples, but the present invention is not limited thereto.

In the following Examples and Experimental examples, MKC-242 indicates 5-[3-[[(2S)-1,4-benzodioxan-2-ylmethyl]amino]propoxy]-1,3-benzodioxole hydrochloride (hydrochloride of a compound of Compound No. 1).

### Example 1

300 g of MKC-242, 1500 g of D-mannitol (trade name: D-Mannitol, Kao Corporation), 900 g of hydrogenated oil (trade name: Lubri Wax 101, Kawaken Fine Chemicals. Co., Ltd.), 150 g of Microcrystalline cellulose (trade name: Avicel PH101, Asahi Chemical Industry Co., Ltd.) and 150 g of hydroxypropylcellulose (trade name: HPC-L, Nippon Soda Co., Ltd.) are placed into a stirring and mixing granulator (FS-GS-25J type, Fukae Kogyo) to mix them. Then, 390 g of water is added while mixing, followed by kneading. This kneaded material is granulated by extruding with a cylindrical granulator (HG-200 type, Hata Tekkosho). A size of the resulting extruded granule is adjusted with Malmerizer (Q-230 type, Dalton Corporation), and the granule is placed into a fluidized bed granulating dryer (FLO-5M type, Freund Industrial Co., Ltd.) and is dried with a warm window at 70?C.

Then, 1000 g of the resulting granule is placed into a rolling fluidized bed granulating dryer (MP-01 type, Powrex Corporation), and a coating solution obtained by adding 555 g of methacrylic acid copolymer LD (trade name: Eudragit L30D-55, Degussa), 17 g of triethyl citrate (trade name: Citroflex SC60, Morimura Bros., Inc.), 17 g of talc (Hayashi Kasei Inc.) and 555 g of water is sprayed thereto while blowing a warm wind at 60?C therein, to obtain a granule in which drug release is regulated.

### Example 2

300 g of MKC-242, 1500 g of D-mannitol (trade name: D-Mannitol, Kao Corporation), 900 g of carnauba wax (trade name: Polishing wax 103, Kawaken Fine Chemicals. Co., Ltd.), 150 g of Microcrystalline cellulose (trade name: Avicel PH101, Asahi Chemical Industry Co., Ltd.) and 150 g of hydroxypropylcellulose (trade name HPC-L, Nippon Soda Co., Ltd.) are placed into a stirring and mixing granulator (FS-GS-25J type, Fukae Kogyo) to mix them. Then, 390 g of water is added while mixing, followed by kneading. This kneaded material is granulated by extruding with a cylindrical granulator (HG-200 type, Hata Tekkosho). A size of the resulting extruded granule is adjusted with Malmerizer (Q-230-type, Dalton Corporation), and the granule is placed into a fluidized bed granulating dryer (FLO-5M type, Freund Industrial Co., Ltd.) and is dried with a warm wind at 70?C.

Then, 1000 g of the resulting granule is placed into a rolling fluidized bed granulating dryer (MP-01 type, Powrex Corporation), and a coating solution obtained by adding 555 g of methacrylic acid copolymer (LD (trade name: Eudragit L30D-55, Degussa), 17 g of triethyl citrate (trade name: Citroflex SC60, Morimura Bros., Inc.), 17 g of talc (Hayashi Kasei Inc.) and 555 g of water is sprayed thereto while blowing a warm wind at 60?C therein, to obtain a granule in which drug release is regulated.

### Example 3

300 g of MKC-242, 1500 g of D-mannitol (trade name: D-Mannitol, Kao Corporation), 900 g of stearic acid (trade name: Stearic Acid, Kao Corporation), 150 g of Microcrystalline cellulose (trade name: Avicel PH101, Asahi Chemical Industry Co., Ltd.) and 150 g of hydroxypropylcellulose (trade name: HPC-L, Nippon Soda Co., Ltd.) are placed into a stirring and mixing granulator (FS-GS-25J type, Fukae Kogyo) to mix them. Then, 390 g of water is added while mixing, followed by kneading. This kneaded material is granulated by extruding with a cylindrical granulator (HG-200 type, Hata Tekkosho). A size of the resulting extruded granule is adjusted with Malmerizer (Q-230 type, Dalton Corporation), and the granule is placed into a fluidized bed granulating dryer (FLO-5M type, Freund Industrial Co., Ltd.) and dried with a warm wind at 70?C.

Then, 1000 g of the resulting granule is placed into a rolling fluidized bed granulating dryer (MP-01 type, Powrex Corporation), and a coating solution obtained by adding 555 g of methacrylic acid copolymer LD (trade name: Eudragit L30D-55, Degussa), 17 g of triethyl citrate (trade name: Citroflex SC60, Morimura Bros., Inc.), 17 g of talc (Hayashi Kasei Inc.) and 555 g of water is sprayed thereto while blowing a warm wind at 60?C therein, to obtain a granule in which drug release is regulated.

### Example 4

300 g of MKC-242, 1800 g of D-mannitol (trade name: D-Mannitol, Kao Corporation), 600 g of hydrogenated oil (trade name: Lovely Wax 101, Kawaken Fine Chemicals. Co., Ltd.), 150 g of Microcrystalline cellulose (trade name: Avicel PH101, Asahi Chemical Industry Co., Ltd.) and 150 g of hydroxypropylcellullose (trade name: HPC-L, Nippon Soda Co., Ltd.) are placed into a stirring and mixing granulator (FS-GS-25J type, Fukae Kogyo). Then, 390 g of water is added while mixing, followed by kneading. This kneaded material is granulated by extruding with a cylindrical granulator (HG-200 type, Hata Tekkosho). A size of the resulting extruded granule is adjusted with Malmerizer (Q-230 type, Dalton Corporation), and the granule is placed into a fluidized bed granulating dryer (FLO-5M type, Freund Industrial Co., Ltd.) and is dried with a warm wind at 70?C.

Then, 1000 g of the resulting granule is placed into a rolling fluidized bed granulating dryer (MP-01 type, Powrex Corporation), and a coating solution obtained by adding 555 g of methacrylic acid copolymer LD (trade name: Eudragit L30D-55, Degussa), 17 g of triethyl citrate (trade name: Citroflex SC60, Morimura Bros., Inc.), 17 g of talc (Hayashi Kasei Inc.) and 555 g of water is sprayed thereto while blowing a warm wind at 60?C therein, to obtain a granule in which drug release is regulated.

### Comparative Example 1

### Conventional Preparation

40 g of MKC-242, 4656 g of D-mannitol (trade name: D-Mannitol, Kao Corporation), and 540 g of sodium carboxymethylstarch (trade name: Primojel, Matsutani Chemical Industry Co., Ltd.) are placed into a stirring and mixing granulator (FS-GS-25J of type, Fukae Kogyo) to mix them. Then, the mixed powder is granulated using a 6% dissolving solution containing hydroxypropylcellulose (trade name: HPC-L, Nippon Soda Co. Ltd.) and water by a fluidized bed granulator (FLO-5M, Freund Industrial Co., Ltd.), to obtain a granule. A size of this resulting granulated granule is adjusted with a size adjuster (ND-10, Okada Seiko). Further, 5255 g of this size-adjusted powder and 55 g of magnesium stearate (trade name: Magnesium Stearate, Nitto Kasei Co., Ltd.) are mixed with a V-type mixer (SVM-50, Meiwa Co., Ltd.) to obtain a tablet bulk powder. This tablet bulk powder is made tablet with a tabletting machine (AQUA, Kikusui Seisakusho Ltd.) to obtain a fast-releasing tablet.

### Experimental Example 1

### Releasing Test

Regarding the granule obtained in Example 1, a releasing pattern was compared at the condition of 100 rpm and 37?C by a basket method (USP dissolution test method first method) using a 0.1 mol/L hydrochloric acid solution (pH 1.2) and a hydrochloric acid/trisodium phosphate buffer (pH 6.8).

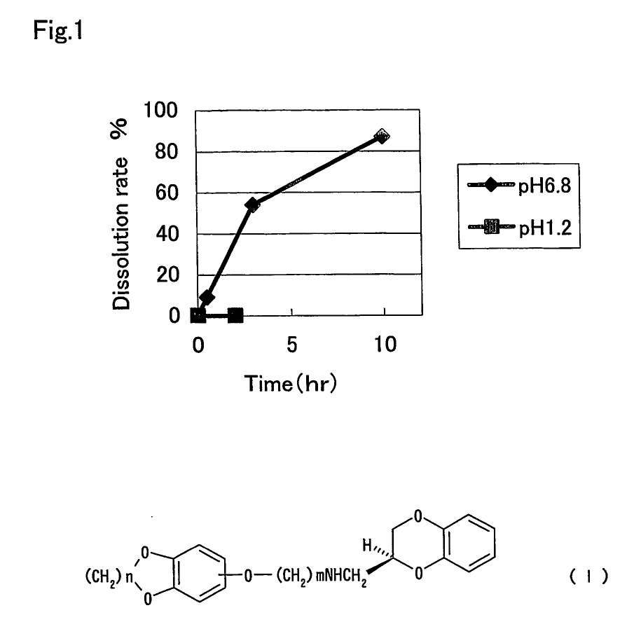

The results are shown in FIG. 1. In the granule obtained in Example 1, release of a drug hardly occurs in the 0.1 mol/L hydrochloric acid solution (pH 1.2) even when two hours passed, and it can be seen that the acid resistance is considerably retained. In addition, in the hydrochloric acid/trisodium phosphate buffer, a 10 hour-type releasing time profile is shown and, in a dissolution test, it was confirmed that a composition for oral administration having a controlled drug releasing rate was obtained.

On the other hand, regarding the conventional preparation obtained in Comparative example 1, a releasing pattern was studied at the condition of 50 rpm and 37?C by a paddling method using Japanese Pharmacopoeia first solution (pH 1.2) and Japanese Pharmacopoeia second solution (pH 6.8). As a result, as shown in FIG. 2, approximate 100% was dissolved out in one hour in the conventional preparation.

### Experimental Example 2

Absorption in a living body and drug acceptability of the granule containing 10 mg of MKC-242 obtained in Example 1 were tested in 6 healthy subjects. As a reference, the conventional preparation (Comparative example 1) having the same drug dose was used.

TABLE 3
Change of drug concentration in blood Unit: ng/ml

| Hour (hr) | Example 1 | Comparative Example 1 |
|---|---|---|
| Predose | 0 | 0 |
| 1 | 2.4 | 335.3 |
| 2 | 51.1 | 255.3 |
| 4 | 178.4 | 101.9 |
| 6 | 79.5 | 36.9 |
| 8 | 50.2 | 20.3 |
| 12 | 30.1 | 7.9 |
| 24 | 10.1 | 1.2 |

TABLE 4
Pharmacokinetic Parameters obtained from unchanged
case concentration in plasma (n = 6)

| | Cₘₐₓ | Tₘₐₓ | T_{1/2} | AUC | Adverse |
|---|---|---|---|---|---|
| Dosage form | (ng/mL) | (hr) | (hr) | (ng*hr/mL) | events |
| Example 1 | 202.4 ? 71.7 | 3.4 ? 0.7 | 11.9 ? 12.6 | 993 ? 242 | 0/6 |
| Comparative Example 1 | 431.8 ? 176.6 | 1.1 ? 0.6 | 2.9 ? 1.1 | 1110 ? 430 | 5/6 |

As seen from Table 3 and Table 4, these two kinds of preparations showed the remarkably different plasma concentration profile. In the regulated-releasing composition of Example 1, the blood concentration is increased at a low absorption rate after around 1 hour lag time as compared with the conventional preparation, and the maximum blood concentration Cₘₐₓ is considerably low as compared with the fast-releasing preparation. In the preparation of Example 1, symptoms such as dizziness was not detected, and the drug acceptability was improved. Further, a half-life T_{1/2} of the drug was extended from 3 hours to 20 hours, demonstrating that the drug concentration in plasma is maintained for a long period of time. In addition, since a scatter of AUC is small, stable drug release is performed in a living body, suggesting the effect of the enteric film.

### INDUSTRIAL APPLICABILITY

The present invention provides a safe and useful pharmaceutical preparation for oral administration which can maintain the drug concentration in plasma for a long period of time, and the composition having the stable drug releasing property in a living body was obtained. Moreover, a drug containing an alkylenedioxybenzene derivative such as MKC-242 and the like can be administered once to twice per day, and a burden of a patient can be alleviated.

1. A composition for oral administration containing an alkylenedioxybenzene derivative represented by the general formula (I): 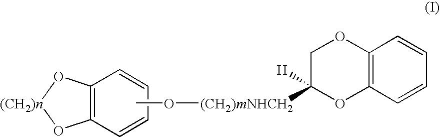
(wherein m represents an integer of 2 to 5, and n represents an integer of 1 to 3) or an acid addition salt thereof and a matrix material and/or a coating material.

2. The composition according to claim 1, wherein each of the matrix material and the coating material is at least one kind selected from a synthetic polymer and waxes.

3. A composition for oral administration, wherein an alkylenedioxybenzene derivative represented by the general formula (I): 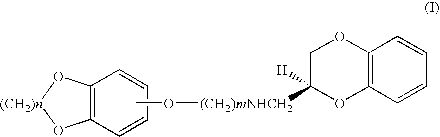
(wherein m represents an integer of 2 to 5, and n represents an integer of 1 to 3) or an acid addition salt thereof is dispersed in a matrix containing a synthetic polymer and/or waxes.

4. A composition for oral administration, characterized in that a composition containing an alkylenedioxybenzene derivative represented by the general formula (I): 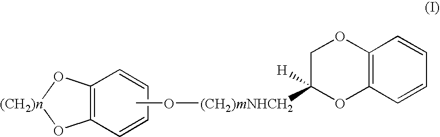
(wherein m represents an integer of 2 to 5, and n represents an integer of 1 to 3) or an acid addition salt thereof and a synthetic polymer and/or waxes is coated with a coating agent containing a synthetic polymer.

5. A composition for oral administration containing an alkylenedioxybenzene derivative represented by the general formula (I): 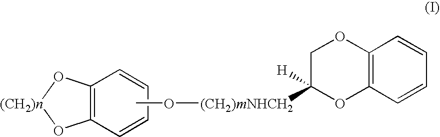
(wherein m represents an integer of 2 to 5, and n represents an integer of 1 to 3) or an acid addition salt thereof in a matrix containing waxes and an excipient.

6. A composition for oral administration, wherein a base granule containing an alkylenedioxybenzene derivative represented by the general formula (I): 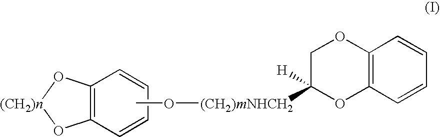
(wherein m represents an integer of 2 to 5, and n represents an integer of 1 to 3)
or an acid addition salt dispersed in a matrix containing waxes and an excipient is coated with an enteric film.

7. The composition for oral administration according to claim 6, wherein an amount of waxes is 5 to 70% by weight relative to the base granule.

8. The composition for oral administration according to claim 1, wherein the synthetic polymer is at least one kind selected from polyvinyl type, acrylic acid or acrylic acid ester type and cellulose type, and the waxes are at least one kind selected from shellac, gelatin, hydrogenated oil, higher fatty acid and esters thereof, higher aliphatic alcohol, and natural and synthetic waxes.

9. A capsule containing an alkylenedioxybenzene derivative or an acid addition salt thereof, comprising the composition for oral administration according to claim 1 filled therein.

10. A process for preparing a composition for oral administration, comprising kneading an alkylenedioxybenzene derivative represented by the general formula (I): 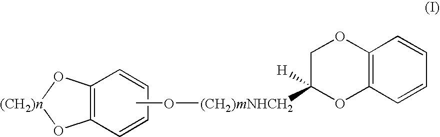
(wherein m represents an integer of 2 to 5, and n represents an integer of 1 to 3) or an acid addition salt thereof, waxes and an excipient to obtain a granule, and coating the granule with an enteric film.

11. A composition for oral administration, wherein a pharmaceutically active substance is an alkylenedioxybenzene derivative represented by general formula (I): 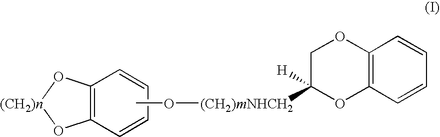
(wherein m represents an integer of 2 to 5, and n represents an integer of 1 to 3) or an acid addition salt thereof, and a time for releasing at least 80% of a content of the pharmaceutically active substance is 2 to 24 hours when tested at 100 rotations per minute using 900 ml of a hydrochloric acid/trisodium phosphate buffer (pH 6.8) as a test solution according to a basket method (USP dissolution test first method).

12. The composition for oral administration according to claim 1, wherein the alkylenedioxybenzene derivative or the acid addition salt thereof is 5-[3-[[(2S)-1,4-benzodioxan-2-ylmethyl]amino]propoxy]-1,3-benzodioxole hydrochloride.

13. The composition for oral administration according to claim 3, wherein the synthetic polymer is at least one kind selected from polyvinyl type, acrylic acid or acrylic acid ester type and cellulose type, and the waxes are at least one kind selected from shellac, gelatin, hydrogenated oil, higher fatty acid and esters thereof, higher aliphatic alcohol, and natural and synthetic waxes.

14. The composition for oral administration according to claim 13, wherein the alkylenedioxybenzene derivative or the acid addition salt thereof is 5-[3-[[(2S)-1,4-benzodioxan-2-ylmethyl]amino]propoxy]-1,3-benzodioxole hydrochloride.

15. The composition for oral administration according to claim 4, wherein the synthetic polymer is at least one kind selected from polyvinyl type, acrylic acid or acrylic acid ester type and cellulose type, and the waxes are at least one kind selected from shellac, gelatin, hydrogenated oil, higher fatty acid and esters thereof, higher aliphatic alcohol, and natural and synthetic waxes.

16. The composition for oral administration according to claim 15, wherein the alkylenedioxybenzene derivative or the acid addition salt thereof is 5-[3-[[(2S)-1,4-benzodioxan-2-ylmethyl]amino]propoxy]-1,3-benzodioxole hydrochloride.

17. The composition for oral administration according to claim 5, wherein the synthetic polymer is at least one kind selected from polyvinyl type, acrylic acid or acrylic acid ester type and cellulose type, and the waxes are at least one kind selected from shellac, gelatin, hydrogenated oil, higher fatty acid and esters thereof, higher aliphatic alcohol, and natural and synthetic waxes.

18. The composition for oral administration according to claim 17, wherein the alkylenedioxybenzene derivative or the acid addition salt thereof is 5-[3-[[(2S)-1,4-benzodioxan-2-ylmethyl]amino]propoxy]-1,3-benzodioxole hydrochloride.

19. The composition for oral administration according to claim 6, wherein the synthetic polymer is at least one kind selected from polyvinyl type, acrylic acid or acrylic acid ester type and cellulose type, and the waxes are at least one kind selected from shellac, gelatin, hydrogenated oil, higher fatty acid and esters thereof, higher aliphatic alcohol, and natural and synthetic waxes.

20. The composition for oral administration according to claim 19, wherein the alkylenedioxybenzene derivative or the acid addition salt thereof is 5-[3-[[(2S)-1,4-benzodioxan-2-ylmethyl]amino]propoxy]-1,3-benzodioxole hydrochloride.
